(19) United States
(12) Plant Patent Application Publication
(10) Pub. No.: US 2016/0174443 P1
(43) Pub. Date: Jun. 16, 2016

(54) Vitex agnus-castus plant named 'V07-2'

(50) ;

(71) Applicant: University of Georgia Research Foundation, Inc., Athens, GA (US)

(72) Inventors: Carol D. Robacker, Peachtree City, GA (US); David A. Knauft, Watkinsville, GA (US)

(73) Assignee: University of Georgia Research Foundation, Inc., Athens, GA (US)

(21) Appl. No.: 14/544,229

(22) Filed: Dec. 10, 2014

(51) Int. Cl. A01H 5/00

(52) U.S. Cl. PLT/226

(57) ABSTRACT

The Vitex agnus-castus plant is of short height, has a compact form and dark pink flowers.

### GENUS AND SPECIES

Vitex agnus-castus.

### VARIETAL DENOMINATION

The new Vitex agnus-castus claimed is of the cultivar denominated ‘V07-2’.

### BACKGROUND OF THE INVENTION

The present invention relates to a new and distinct cultivar of Vitex agnus-castus hereinafter referred to by the varietal domination ‘V07-2’.

Pedigree and history: ‘V07-2’ originated from a cross of Vitex agnus-castus ‘Salinas Pink’ (unpatented, female parent) and Vitex agnus-castus ‘Abbeville Blue’ (unpatented, male parent) made in 2006 under the direction of David Knauft. Seedlings from this cross were grown in a field plot in Watkinsville, Ga. In 2006, a single plant, initially labeled 03-06, was identified and selected. This selected plant had deep pink flowers and an attractive form. Cuttings were made from this plant, and distributed to Carol Robacker, who took over this breeding program in 2009. This selection was given the cultivar name ‘V07-2’, and plants of this cultivar were planted in a replicated field plot (three reps, randomized block design) in Griffin, Ga. in the fall of 2009. ‘V07-2’ has been grown in an irrigated field plot in Griffin, Ga. Plants have been fertilized annually in the spring.

Mean panicle lengths and number of panicles per compound panicle were counted each summer. First bloom and re-bloom dates were noted each year. Data was collected on leaf yellowing, leaf drop, thinning, and leaf spot in July, August and September of each year.

‘V07-2’ has been grown in an irrigated field plot in Griffin, Ga. Plants have been fertilized annually in the spring. The height of these plants after five summers' growth averaged about 2.9 m. Stems are R.H.S. (Royal Horticultural Society, 2001) brown N199A or 199B when older, round, with dense minute curved hairs and scattered longer curved hairs.

The trunk color is a mix of greyed-white 156A and grey-brown 199D. Stem striations begin on large stems or trunks at about 2.5 cm diameter, with cracking and exfoliation noticeable at about 5 cm diameter. Leaves emerge with a green 137C upper surface, changing to a deeper green 137A during the summer, and becoming yellow-green 146A in the fall. Lower leaf surface is greyed-green 191B throughout the season.

Leaves are palmately-compound, typically about 13 cm×14 cm, with mostly seven leaflets. Margins are entire, with acute apices and bases. The upper leaf surface has many scattered hairs, slightly glandular, while the lower surface is more densely pubescent and very glandular. Leaves are slightly waxy above, dull underneath. Venation is simple. Leaf arrangement on stems is opposite.

Flower buds are red-purple 65C. Flowers occur in an elongated compound panicle, 22 to 26 cm long. At emergence, flowers are red-purple N74D, and become a darker red-purple N74C at full bloom. The peduncle is grayed-green 193A. The number of individual flowers per inflorescence ranges from 254 to 632. Sepals are greyed-green 198A.

### SUMMARY OF THE INVENTION

‘V07-2’ plants have been evaluated for four years at the Griffin, Ga. site. Height and width data has been collected annually. Cold damage was assessed each spring. Mean panicle lengths and number of panicles per compound panicle were counted each summer. First bloom and re-bloom dates were noted each year. Data was collected on leaf yellowing, leaf drop, thinning, and leaf spot in July, August and September of each year.

‘V07-2’ plants from cuttings were was planted into a field plot in Griffin, Ga. in June 2009. A ‘Salinas Pink’ was planted into the same plot in June 2011. All data in the Tables are from rooted liners grown in one-gallon containers. Data given are averages of measurements made on three plants of ‘V07-2’ and one plant for ‘Salinas Pink’.

The following characteristics have been consistently observed in the original plant of this new variety and in asexually propagated progeny grown from cuttings and, to the best knowledge of the inventors, their combination forms the unique characteristics of ‘V07-2’ as a new and distinct cultivar. Asexual propagation by cuttings has proven that these characteristics are firmly fixed in succeeding asexually propagated generations.
1. Short height (Table 1);
2. Compact form (FIG. 3);
3. Dark pink flowers (FIG. 2).

‘V07-2’ is an improved pink Vitex as compared to the industry standard ‘Salinas Pink’, the only commonly available pink Vitex.

Comparison. Height and width were measured annually. ‘Salinas Pink’ is a taller and wider plant than ‘V07-2’(Table 1). Both ‘V07-2’ and ‘Salinas Pink’ begin flowering at the same time each year (Table 2). Both have moderate repeat blooming during summer and fall (Table 3). Panicles on ‘V07-2’ are generally a bit shorter, but with greater diameter than those on ‘Salinas Pink’ (Table 4, FIG. 1). Flowers of ‘V07-2’ are a darker pink than those of ‘Salinas Pink’ (FIG. 2). ‘V07-2’ has a rounded, compact form, while ‘Salinas Pink’ is very irregular in growth habit (FIG. 3). ‘Abbeville Blue’ has flowers that have a blue coloration.

‘V07-2’ is an improved pink Vitex, as compared to the industry standard, ‘Salinas Pink’. It is shorter in height (Table 1), has a more compact form (FIG. 3), and a darker pink flower (FIG. 2).

Vitex agnus-castus is a deciduous shrub or small tree used in landscapes. This drought tolerant plant may be grown in cold hardiness zones 6 through 9. During cold winters in zone 6, it may die back to the ground, but will likely re-grow from the roots and produce a flowering shrub during the following summer, as flower buds are formed on new growth. Late spring freezes in zone 7 may also cause cold damage and dieback, but the plants recover and bloom during the summer.

‘V07-2’ has been and is propagated vegetatively by stem cuttings.

TABLE 1
Height and width (cm) of Vitex ‘V07-2’ and ‘Salinas Pink’ one, two
and three years after planting in a field plot in Griffin, Georgia.
Data for ‘V07-2’ is the average of three plants.

| Entry | Year 1 | Year 2 | Year 3 |
|---|---|---|---|
| ‘V07-2’ | 145 H × 222 W | 172 H × 236 W | 245 H × 292 W |
| ‘Salinas Pink’ | 118 H × 142 W | 228 H × 291 W | 297 H × 370 W |

TABLE 2
Flowering characteristics of V07-2 and
‘Salinas Pink’ grown in Griffin, Georgia.

| | First | First | First |
|---|---|---|---|
| | bloom | bloom | bloom |
| Entry | 2011 | 2012 | 2013 |
| ‘V07-2’ | 5-30 | 5-14 | 6-10 |
| ‘Salinas Pink’ | — | 5-14 | 6-10 |

TABLE 3
Repeat flowering in July, August, September and
October in 2011, 2012, and 2013.

| Entry | Year | July | August | September | October |
|---|---|---|---|---|---|
| ‘V07-2’ | 2011 | 0* | 1.7 | 2.2 | 0 |
| | 2012 | 1.3 | 3.0 | 4.0 | 0.7 |
| | 2013 | 0.7 | 0.3 | 1.3 | 1.2 |
| ‘Salinas Pink’ | 2011 | 0 | 1.0 | 3.0 | 2.0 |
| | 2012 | 2.0 | 3.0 | 3.0 | 2.0 |
| | 2013 | 1.0 | 0 | 0.5 | 0.5 |

*Repeat flowering was rated using the following scale:
0 = no flowering;
1 = 10% full bloom;
2 = 20 to 30% full bloom;
3 = 40 to 50% full bloom;
4 = at least 60% full bloom.

TABLE 4
Panicle length, diameter and number of sub-panicles per panicle
averaged over 2011, 2012, and 2013. Measurements were made on
four typical or average-sized panicles per plant per year.

| | Mean | Mean | |
|---|---|---|---|
| | Panicle | Panicle | Number of |
| Entry | length (cm) | diameter (cm) | subpanicles/panicle |
| ‘V07-2’ | 22.5 | 4.4 | One long, two to four short |
| ‘Salinas Pink’ | 24.1 | 3.2 | One long, two to four short |

### BRIEF DESCRIPTION OF THE FIGURES

The accompanying colored photographic illustrations show the overall appearance and distinct characteristics of the new cultivar of Vitex agnus-castus. The colors in the photographs are as close as possible with the photographic and printing technology utilized.

FIG. 1 is a photograph of the new variety ‘V07-2’ (left image) during flowering in Griffin, Ga. and a photograph of ‘Salinas Pink’ (right image). ‘V07-2’ has darker pink flowers with a wider diameter than ‘Salinas Pink’. ‘Salinas Pink’ has slighter longer panicles.

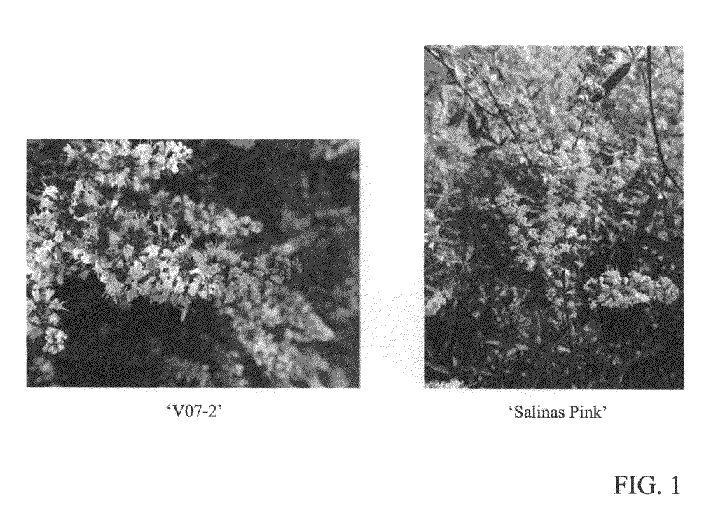

FIG. 2 is a close up of the new variety ‘V07-2’ during flowering in Griffin, Ga. on the left, and ‘Salinas Pink’ on the right, taken from flower clusters of similar blooming stage.

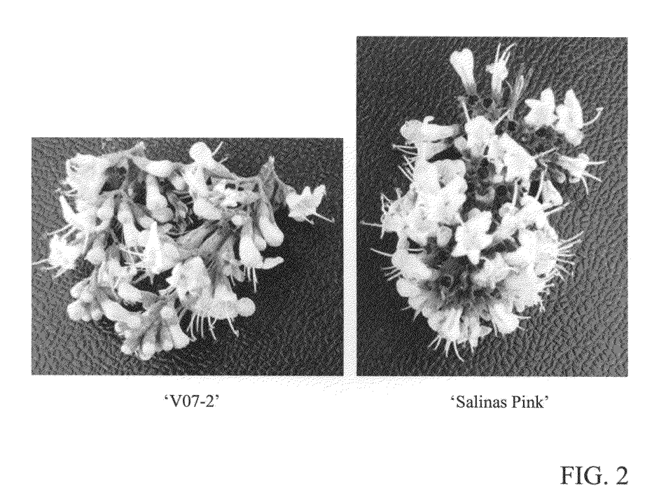

FIG. 3 is a photograph of ‘V07-2’ (left image) and ‘Salinas Pink’ (right image) in peak bloom in Griffin, Ga. The form on ‘V07-2’ is more compact and rounded, while ‘Salinas Pink’ has an irregular form.

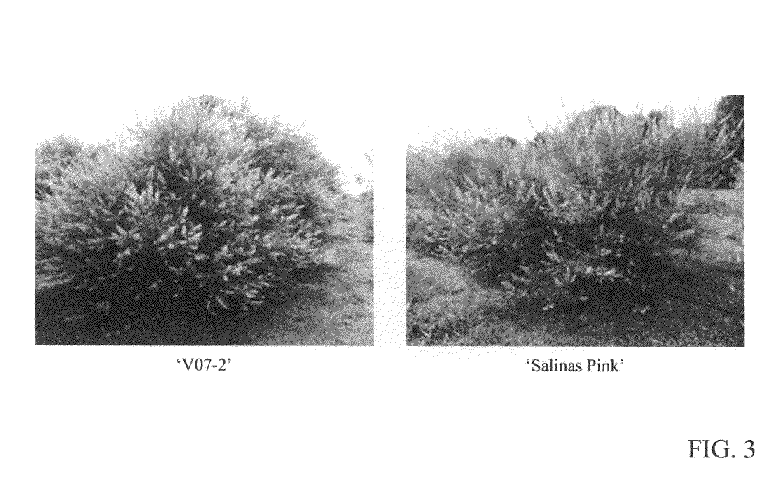

### DETAILED BOTANICAL DESCRIPTION

The following is a detailed description of the Vitex agnus-castus cultivar named ‘V07-2’. Data was collected in Griffin, Ga. from three year old plants grown from cuttings and growing outdoors. ‘V07-2’ has not been tested under all possible conditions hence, phenotypic differences may be observed with variations in environmental conditions without any variance in genotype.

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society (R.H.S.), London, England. This description is from observations of typical three year old plants growing in Griffin, Ga.
Parentage: Female parent — ‘Salinas Pink’Male parent — ‘Abbeville Blue’
Size: 245 cm tall by 292 cm wide, measured at highest and widest point. Size is reflective of habit, i.e., 2′ by 3′ is rounded to broad-rounded.
Habit: Open, spreading, upright.
Texture: Medium coarse.
Stems:
   First year.—Color: Grey-Brown N199A Diameter: 4-5 mm Pubescence: dense minute curved hairs, scattered longer curved hairs Exfoliation: none Shape: Round Pith: Type: Solid Diameter: 3 mm Color: Yellow-Green 150D Odor: strong, spicy, acrid Internode Length: 6.5 cm.
   Second year.—Color: Mix of Grey-Brown 199B and Brown 200N Diameter: approx. 6 mm Exfoliation: none.
Vegetative buds:
   Arrangement.—opposite.
   Type.—valvate.
   Size.—1 mm×1 mm.
   Scale number.—2.
   Scale color.—Greyed-Orange 177C.
   Position/disposition.—45°.
   Number at node.—2, one on each side.
   Pubescence.—scattered short hairs.
   Shape.—domed, rounded, mostly covered by bud scales.
Leaf scar:
   Shape.—cup shaped.
   Vascular bundle traces.—3, horizontal.
   Pubescence.—dense minute airs around perimeter.
   Position of bud.—just above.
   Color differentiation.—Yellow-Green 144A.
   Size.—2×3 mm.
Trunk or large stems:
   Color(s).—Mix of Greyed-White 156A and Greyed-Brown 199D.
   Size stem exfoliation begins on.—Approximately 5 cm.
   Diameter.—2.5 to 5.0 cm.
   Texture.—striations start at 2.5 cm, cracking by 5.0 cm.
Leaf:
   Color through seasons.—Emerging dates — mid April Upper: Green 137C Lower: Greyed-Green 191 B Summer dates — mid July Upper: Green 137A Lower: Greyed-Green 191 B Fall dates — September Upper: Yellow-Green 146A Lower: Yellow-Green 191 B.
   Mature size.—13 cm×14 cm.
   Apex.—Acute.
   Base.—Acute.
   Margin.—mostly entire.
   Shape.—palmate, mostly 7 leaflets. Lobes: None Sinuses: none.
   Vein color.—Yellow-Green 147D.
   Pubescence.—Upper surface has many scattered hairs, with more along the veins, slightly grandular surface. Lower leaf is more densely hairy and has a very grandular surface.
   Arrangement on stem.—Opposite.
   Venation.—Simple.
   Texture.—Thickness: 0.4 mm Degree of waxiness of surfaces: slightly waxy on upper surface, dull on lower.
Petiole:
   Length.—4.3 cm.
   Shape.—round.
   Color.—upper Greyed-Orange 177A, lower Yellow-Green 145B with some Greyed-Orange N167A.
   Pubescence.—densely covered in minute slightly curved hairs.
   Diameter.—1.7 mm.
Flower buds:
   Size (l×w).—5 mm×3.5 mm.
   Color.—Red-Purple 65C.
   Shape.—teardrop.
   Pubescence.—glandular surface, tomentose.
   Time of full maturity (first visible).—early summer.
   Time range for showiness.—mid May through September.
Flower:
   Inflorescence(s).—Type: elongated panicle, compound, slightly whorled Size (l×w): 22-26 cm×2.0-4.4 cm Color: at emergence (date) — Red-Purple N74D full bloom (date) — Red-Purple N74C fading (date) — Red-Purple 63D with Red-Purple N74B Peduncle: Color: Greyed-Green 193A Pubescence: glandular, tomentose Number of individual flowers per inflorescence: 254-545, 315-632.
   Petal(s).—Size: 10 mm×8 mm Shape: zygomorphic, gamopetalous, bilabiate Apex: 5 lobes rounded and slightly curled Base: funnel Margin: slightly curled Pubescence: mostly glabrous, scattered glandular with thick tuft of hairs inside base and many glandular hairs outside Texture: mostly smooth, slightly punctate Color at peak of bloom: upper surface — Red-Purple N74C lower surface — Red-Purple 65C Pedicels: color (RHS) — Greyed-Green 193A pubescence — glandular, tomentose length — 2-3 mm.
   Sepal(s).—Size (l×w): 3 mm×2 mm Shape: united, slightly lobed Apex: slightly lobed Base: united, short tubular Margin: smooth Pubescence: glandular, tomentose, canescent Texture: hoary Color at peak of bloom: upper surface — Greyed-Green 198A with traces of Violet-Blue 94A lower surfaces — lower Yellow-Green 144C with Violet-Blue 93C.
   Male reproductive structures.—Number: 4 Anther: size (l×w) — 1 mm×0.5 mm color — White 155C Filament: size (l×w) — 5.5 mm×0.5 mm color — Red-Purple 69D Pollen color — White 155C Pubescence — scattered short hairs with thick tuft at base.
   Female reproductive structures.—Pistil: shape — tubular, bifid size (l×w) — 6 mm×0.5 mm position (superior, inferior, etc.) — superior color (RHS) — Violet 84B pubescence — thickly tufted hairs at base Stigma: shape — round, bifid color (RHS) — White 155C pubescence — none Style: length — 6 mm shape — tubular, forked at stigma (bifid) color (RHS) — Violet 84B pubescence — glabrous, tufted at base Ovary: shape — round number — 1 pubescence — scattered short hairs, numerous glands present on surface.
Fruit:
   Type.—drupe-like.
   Size (l×w).—4 mm×3 mm.
   Color(s) during ripening.—early (date) — Yellow-Green 146D mid (date) — Grey-Brown 199A and Grey-Brown 199B late (date) — Brown 200D and Brown 200A
   Shape.—globular.
   Number per infructescence.—1.
   Pubescence.—few scattered hairs, surface lightly punticulate.
   Number of carpels.—2.
   Persistence (effective period).—mid to late summer through fall into winter.
Seed:
   Shape.—globular.
   Size.—4 mm×3 mm.
   Color.—Brown 200A when fully ripe.
   Number per locule per ovary per fruit.—1.
   Germination capacity.—≈30%.
   Pubescence.—scattered minute hairs.

What is claimed is:

1. A new and distinct cultivar of the Vitex agnus-castus plant named ‘V07-2’ substantially as illustrated and described herein.
